(19) United States
(12) Patent Application Publication
(10) Pub. No.: US 2004/0126415 A1
(43) Pub. Date: Jul. 1, 2004

(54) Dermal delivery of a water-soluble selective cyclooxygenase-2 inhibitor

(76) Inventors: Guang Wei Lu, Ann Arbor, MI (US); Gary D. Ewing, Kalamazoo, MI (US); Brenda M. Stoller, Ypsilanti, MI (US); Kathryn M. Kienle, Kalamazoo, MI (US)

Correspondence Address: PHARMACIA CORPORATION, Global Patent Department, 575 Maryville Cenre Drive, Mail Zone 1006, St. Louis, MO, 63141, US

(21) Appl. No.: 10/683,943

(22) Filed: Oct. 10, 2003

Related U.S. Application Data
(60) Provisional application No. 60/428,201, filed on Nov. 21, 2002.

(51) Int. Cl. A61K 31/42; A61K 9/70

(52) U.S. Cl. 424/449; 514/378

(57) ABSTRACT

A pharmaceutical composition for application to an area of skin of a subject for local and/or systemic treatment of a COX-2 mediated disorder comprises a backing sheet that is flexibly conformable to the area of skin, the backing sheet having opposing surfaces that are respectively distal and proximal to the skin when applied; and a coating on the proximal surface of the backing sheet that comprises (a) an adhesive and (b) a water-soluble active agent selected from selective COX-2 inhibitory drugs, prodrugs and salts thereof, the active agent being in a therapeutically effective total amount and dispersed in a matrix that comprises zero to less than an active agent solubilizing effective amount in total of one or more solvents other than the adhesive. A method of local treatment of a site of pain and/or inflammation in a subject comprises applying the composition to a skin surface of the subject, preferably at a locus overlying or adjacent to the site of pain and/or inflammation, and leaving the composition in place for a time period effective to permit delivery of a locally therapeutic amount of the active agent. A method of systemic treatment of a subject having a COX-2 mediated disorder comprises applying the composition to a skin surface of the subject, and leaving the composition in place for a time period effective to permit transdermal delivery of a therapeutic amount of the active agent.

This application claims priority of U.S. provisional application Serial No. 60/428,201 filed on Nov. 21, 2002.

### FIELD OF THE INVENTION

The present invention relates to pharmaceutical compositions containing a water-soluble selective cyclooxygenase-2 (COX-2) inhibitory drug, in particular to such compositions in adhesive coated sheet form that are suitable for administration to skin to provide a local or systemic therapeutic effect. An “adhesive coated sheet” herein includes patches, tapes, poultices, pads, plasters, cataplasms, dressings and the like. The invention also relates to processes for preparing such compositions and to methods of treatment comprising administration of such compositions to skin of a subject in need thereof.

### BACKGROUND OF THE INVENTION

Inhibition of cyclooxygenase (COX) enzymes is believed to be at least the primary mechanism by which nonsteroidal anti-inflammatory drugs (NSAIDs) exert their characteristic anti-inflammatory, antipyretic and analgesic effects, through inhibition of prostaglandin synthesis. Conventional NSAIDs such as ketorolac, diclofenac, naproxen and salts thereof inhibit both the constitutively expressed COX-1 and the inflammation-associated or inducible COX-2 isoforms of cyclooxygenase at therapeutic doses. Inhibition of COX-1, which produces prostaglandins that are necessary for normal cell function, appears to account for certain adverse side effects that have been associated with use of conventional NSAIDs. By contrast, selective inhibition of COX-2 without substantial inhibition of COX-1 leads to anti-inflammatory, antipyretic, analgesic and other useful therapeutic effects while minimizing or eliminating such adverse side effects. Selective COX-2 inhibitory drugs have therefore represented a major advance in the art.

Numerous compounds have been reported having therapeutically and/or prophylactically useful selective COX-2 inhibitory effect, and have been disclosed as having utility in treatment or prevention of specific COX-2 mediated disorders or of such disorders in general. Among such compounds are a large number of substituted pyrazolyl benzenesulfonamides as reported in U.S. Pat. No. 5,466,823 to Talley et al., including for example the compound 4-[5-(4-methylphenyl)-3-(trifluoromethyl)-1H-pyrazol-1-yl]benzenesulfonamide, also referred to herein as celecoxib (I), and the compound 4-[5-(3-fluoro-4-methoxyphenyl)-3-difluoromethyl)-1H-pyrazol-1-yl]benzenesulfonamide, also referred to herein as deracoxib (II).

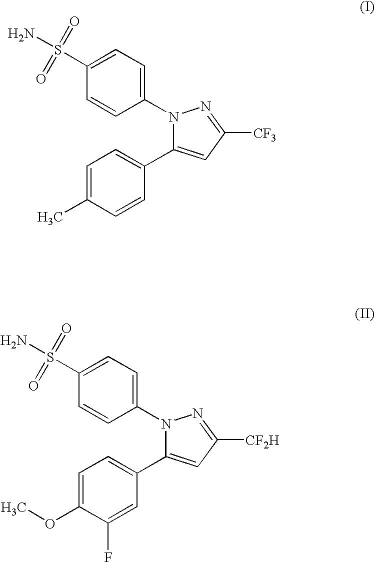

Other compounds reported to have therapeutically and/or prophylactically useful selective COX-2 inhibitory effect are substituted isoxazolyl benzenesulfonamides as reported in U.S. Pat. No. 5,633,272 to Talley et al., including for example the compound 4-[5-methyl-3-phenylisoxazol-4-yl]benzenesulfonamide, also referred to herein as valdecoxib (III).

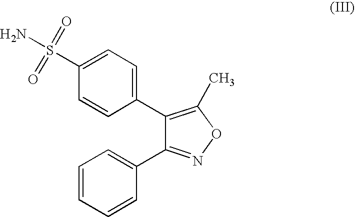

Still other compounds reported to have therapeutically and/or prophylactically useful selective COX-2 inhibitory effect are substituted (methylsulfonyl)phenyl furanones as reported in U.S. Pat. No. 5,474,995 to Ducharme et al., including for example the compound 3-phenyl-4-[4-(methylsulfonyl)phenyl]-5H-furan-2-one, also referred to herein as rofecoxib (IV).

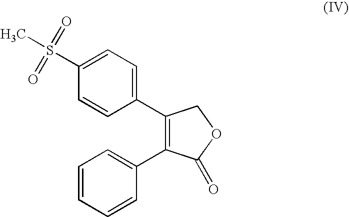

U.S. Pat. No. 5,981,576 to Belley et al. discloses a further series of (methylsulfonyl)phenyl furanones said to be useful as selective COX-2 inhibitory drugs, including 3-(1-cyclopropylmethoxy)-5,5-dimethyl-4-[4-(methylsulfonyl)phenyl]-5H-furan-2-one and 3-(1-cyclopropylethoxy)-5,5-dimethyl-4-[4-(methylsulfonyl)phenyl]-5H-furan-2-one.

U.S. Pat. No. 5,861,419 to Dube et al. discloses substituted pyridines said to be useful as selective COX-2 inhibitory drugs, including for example the compound 5-chloro-3-(4-methylsulfonyl)phenyl-2-(2-methyl-5-pyridinyl)pyridine, also referred to herein as etoricoxib (V).

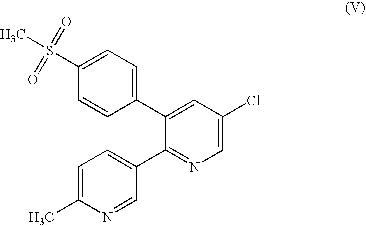

European Patent Application No. 0 863 134 discloses the compound 2-(3,5-difluorophenyl)-3-[4-(methylsulfonyl)phenyl]-2-cyclopenten-1-one said to be useful as a selective COX-2 inhibitory drug. International Patent Publication No. WO 99/11605 discloses 5-alkyl-2-arylaminophenylacetic acids and derivatives thereof, including the compound 5-methyl-2-(2′-chloro-6′-fluoroanilino)phenylacetic acid and salts thereof, said to be selective inhibitors of COX-2.

U.S. Pat. No. 6,034,256 to Carter et al. discloses a series of benzopyrans said to be useful as selective COX-2 inhibitory drugs, including the compound (S)-6,8-dichloro-2-(trifluoromethyl)-2H-1-benzopyran-3-carboxylic acid (VI)

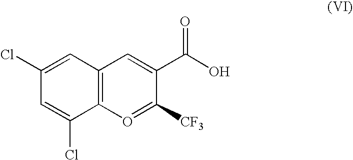

the compound (S)-6-chloro-7-(1,1 -dimethylethyl)-2-(trifluoromethyl)-2H-1-benzopyran-3-carboxylic acid (VII)

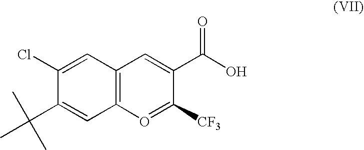

and salts thereof

International Patent Publication No. WO 00/24719 discloses substituted pyridazinones said to be useful as selective COX-2 inhibitory drugs, including the compound 2-(3,4-difluorophenyl)-4-(3-hydroxy-3-methyl-1-butoxy)-5-[4-(methylsulfonyl)phenyl]-3-(2H)-pyridazinone.

Selective COX-2 inhibitory drugs have been formulated in a variety of ways, principally for oral delivery. However, topical administration of such drugs has been suggested in general terms, for example in some of the above-cited patents.

Above-cited U.S. Pat. Nos. 5,466,823 and No. 5,633,272 disclose that their subject compounds, which include celecoxib and valdecoxib, can be delivered topically.

Above-cited U.S. Pat. No. 5,474,995 discloses that its subject compounds, which include rofecoxib, can be formulated as creams, ointments, jellies, solutions or suspensions for topical use. Above-cited U.S. Pat. No. 5,861,419 similarly discloses that its subject compounds, which include etoricoxib, can be formulated as creams, ointments, jellies, solutions or suspensions for topical use, and further suggests that topical formulations may generally be comprised of a pharmaceutical carrier, co-solvent, emulsifier, penetration enhancer, preservative system and emollient.

U.S. Pat. No. 5,932,598 to Talley et al. discloses a class of water-soluble prodrugs of selective COX-2 inhibitory drugs, including the compound N-[[4-(5-methyl-3-phenylisoxazol-4-yl)phenyl]sulfonyl]propanamide, also referred to herein as parecoxib (VIII), and salts thereof, for example the sodium salt, referred to herein as parecoxib sodium. Parecoxib converts to the substantially water-insoluble selective COX-2 inhibitory drug valdecoxib following administration to a subject. Parecoxib itself shows weak in vitro inhibitory activity against both COX-1 and COX-2, while valdecoxib (II) has strong inhibitory activity against COX-2 but is a weak inhibitor of COX-1.

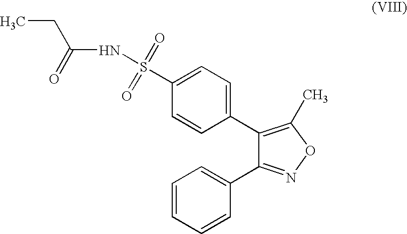

Because of the high water solubility of parecoxib, particularly of salts such as parecoxib sodium, by comparison with most selective COX-2 inhibitory drugs such as celecoxib and valdecoxib, the prodrug parecoxib has been proposed for parenteral use. See Talley et al. (2000), J. Med. Chem. 43, 1661-1663.

Above-cited U.S. Pat. Nos. 5,932,598 and No. 6,034,256 disclose that their subject compounds can be administered by a transdermal device, for example using a patch either of the reservoir and porous membrane type or of a solid matrix variety. In either case, the active agent is said to be delivered continuously from the reservoir or microcapsules through a membrane into an adhesive that is permeable to the active agent, the adhesive being in contact with the skin or mucosa of the recipient.

U.S. Pat. No. 5,208,035 to Ikeda et al. discloses a plaster comprising a backing material and a paste spread thereon. The paste comprises the NSAID diclofenac sodium, 1-menthol, propylene glycol and a water-soluble polymer.

U.S. Pat. No. 5,591,767 to Baker et al. discloses a transdermal patch having a depot of the NSAID ketorolac between an occlusive backing layer and a porous membrane. The depot contains, in addition to the ketorolac, a plasticizing-type enhancer selected from isopropyl myristate, caprylic triglyceride, capric triglyceride and glyceryl oleate, and a solvent-type enhancer selected from ethanol, propanol and propylene glycol. An adhesive layer is in contact with the skin-facing side of the porous membrane.

U.S. Pat. No. 5,607,690 to Akizawa discloses an anti-inflammatory and analgesic plaster preparation containing the NSAID diclofenac in the form of its hydroxyethylpyrrolidine salt, which is reported to exhibit enhanced skin permeation by comparison with an otherwise similar preparation containing diclofenac sodium. The low skin permeability of diclofenac sodium is stated therein to result from the low solubility in water of this salt.

U.S. Pat. No. 5,665,378 to Davis & Primo-Davis discloses a transdermal patch formulation comprising an NSAID, the diuretic drug pamabroom, capsaicin and a skin permeation enhancer selected from menthol, eucalyptol, glyceryl monostearate and d-limonene. The formulation is said to be useful for treating menstrual pain.

U.S. Pat. No. 5,916,587 to Jeong et al. discloses a transdermal patch having an adhesive polymer matrix containing the NSAID piroxicam, an absorption assistant (typically a solvent) and a penetration enhancer.

Japanese Patent Application No. 06-219940 discloses a transdermal patch having a reservoir comprising the NSAID diclofenac sodium in an oil-in-water emulsion.

International Patent Publication No. WO 94/23713 discloses a topical and/or transdermal delivery composition comprising an NSAID, illustratively flurbiprofen, a lipophilic excipient selected from fatty acid alkyl esters and monoglycerides, and a hydrophilic excipient selected from polyethylene glycol, polyethylene glycol esters, isosorbide ethers and diethylene glycol ethers. A pressure sensitive adhesive can be included in the formulation for application to a flexible backing, to form an adhesive-coated sheet material useful as a tape, patch or dressing.

International Patent Publication No. WO 97/29735 discloses a transdermal drug delivery system comprising a dermal penetration enhancer that is an ester sunscreen, preferably a long-chain alkyl ester of p-aminobenzoic acid, dimethyl p-aminobenzoic acid, cinnamic acid, methoxycinnamic acid or salicylic acid, for example octyl dimethyl p-aminobenzoate or octyl salicylate.

Japanese Patent Application No. 10-114646 discloses a patch comprising an illustratively indomethacin, and berberine as an agent to reduce skin irritation.

Japanese Patent Application No. 10-218793 discloses an adhesive tape comprising a styrene-isoprene-styrene block copolymer, the NSAID felbinac, 1-menthol and oleyl alcohol.

Japanese Patent Application No. 10-298065 discloses an adhesive tape said to be “warm-feeling”, prepared by laminating a polymer film with a fabric to form a support layer and then laminating with a hydrophilic layer that can contain a blood circulation promoter and an NSAID.

Japanese Patent Application No. 10-298069 discloses a patch comprising an elastic support having thereon a pressure-sensitive adhesive layer that contains polyether-ester-amide adhesives and an NSAID, illustratively ketoprofen.

Japanese Patent Application No. 11-199515 discloses a patch comprising an NSAID selected from flurbiprofen, felbinac, bufexamac and suprofen, one or more water-soluble polymers and two or more multivalent metal compounds.

Japanese Patent Application No. 11-199516 discloses a patch comprising the NSAID flurbiprofen, red pepper extract and a mixture of polymers.

Japanese Patent Application No. 11-199518 discloses a patch comprising the flurbiprofen, red pepper extract and β-cyclodextrin.

Japanese Patent Application No. 11-199519 discloses a patch comprising the NSAID flurbiprofen, red pepper extract and gelatin.

International Patent Publication No. WO 99/62557 discloses a composition for transdermal administration of an NSAID comprising an absorption promoter that consists essentially of a diethylene glycol ether and a sorbitan ester, and an adhesive matrix.

International Patent Publication No. WO 00/41538 discloses a composition for transdermal administration of a drug comprising a blend of two or more acrylic-based polymers having differing functionalities.

International Patent Publication No. WO 00/51575 discloses a transdermal device containing a composition of an NSAID with a skin permeation enhancer selected from fatty alcohols, e.g., oleyl alcohol and fatty acid esters, e.g., glyceryl monooleate, isopropyl myristate.

Japanese Patent Application No. 2000/256214 discloses a patch comprising an NSAID and a thermal sense stimulant selected from red pepper extracts, capsaicin and nonanoic acid vanillylamide, formulated in an adhesive base on a silicone-treated polyester film with a polyethylene fabric layered on top.

Korean Patent Application No. 2000/24702 discloses a poultice comprising the NSAID loxoprofen together with adhesive polymers, auxiliary agents and an absorption accelerator.

European Patent Application No. 1 148 106 discloses a pressure sensitive adhesive tape preparation comprising a drug, e.g., an NSAID, a polyhydric alcohol and a sodium, magnesium, zinc or aluminum salt of a fatty acid.

European Patent Application No. 1 170 020 discloses a composition comprising an NSAID, illustratively diclofenac sodium, and a local anesthetic, illustratively lidocaine, for topical treatment of inflammatory pain, e.g., lumbago. The active agents are reportedly incorporated into an adhesive gel base containing a water-soluble polymer, a crosslinking agent, water and a water holding agent; the gel base is then applied to a nonwoven fabric which is pressed and covered with a polypropylene liner for cutting into patches.

U.S. Pat. No. 6,262,121 to Kawaji & Yamaji discloses an oily patch comprising the NSAID diclofenac sodium, isostearic acid, a fatty acid that is liquid at ambient temperature and an adhesive base.

International Patent Publication No. WO 01/91743 discloses a patch containing, by weight, 0.1-20% of the NSAID 4-biphenylacetic acid (felbinac) together with 5-50% of a styrene/isoprene/styrene block copolymer, 0.05-20% N-methyl-2-pyrrolidone and 0.1-20% polyethylene glycol.

United Kingdom Patent Application No. 2 362 825 discloses a transdermal patch comprising an NSAID, an alkylpyrrolidone, polyethylene glycol and a hydrophilic nonionic surfactant in an aqueous base that comprises a water-soluble polymer, a water-soluble vinyl polymer and a water-insoluble multivalent metallic salt.

Japanese Patent Application No. 2002/193793 discloses patch formulations comprising an NSAID such as flurbiprofen. The formulation is prepared by dissolving or dispersing a glycol in a glycerin-containing gel and dispersing the NSAID into the same gel. The gel is then spread on an elastic nonwoven fabric and covered with a polypropylene film to provide a patch.

International Patent Publication No. WO 02/58620 discloses pharmaceutical compositions containing a COX-2 inhibitor, for example a selective COX-2 inhibitor, and a muscle relaxant, illustratively pridinol mesylate. A wide variety of dosage forms is contemplated therein, including a poultice (emplasto) and a patch (parche).

As the foregoing indicates, administration of an adhesive coated sheet comprising an NSAID, in some cases a selective COX-2 inhibitory drug, to the skin with the objective of achieving local or systemic therapeutic effect has been widely contemplated in the art. However, there remains a need in the art for an adhesive coated sheet composition of a selective COX-2 inhibitory drug that can be shown to exhibit a sufficient rate of skin permeation of the drug to achieve such effect.

Where a systemic effect is desired, the composition must be capable of delivering daily an amount of the drug by skin permeation at least equal to the minimum therapeutically effective daily dosage amount when the drug is given orally or parenterally. Even if bioavailability by the transdermal route is high, this can be a difficult challenge especially where the therapeutic dose is high. Furthermore, it is neither practical nor convenient to apply an adhesive coated sheet to a very large area of skin to achieve this result; typically a maximum area for application to an adult human subject is about 400 cm², but preferably a much smaller area of skin is treated.

For illustration, in the case of celecoxib, a typical minimum daily dosage amount by oral administration for an adult human is about 200 mg. A minimum permeation rate of 500 μg/cm².day over an area of 400 cm² is therefore needed to provide the minimum daily dosage amount of celecoxib. It is generally desirable to treat a much smaller area than 400 cm², thus the minimum permeation rate desired is even higher than 500 μg/cm².day. Even where only local delivery is desired, a high permeation rate is still important, because the area of skin available for local application, for example by patch, poultice or tape, is generally no greater than about 140 cm², often less.

Whether a systemic or local therapeutic effect is desired, it has therefore remained a difficult challenge to formulate a selective COX-2 inhibitory drug in a form of an adhesive coated sheet providing sufficient permeation to provide therapeutic effectiveness, especially when applied to an area of skin no greater than about 400 cm². More specifically, it is a difficult challenge to formulate a water-soluble selective COX-2 inhibitory drug or prodrug in such a form.

### SUMMARY OF THE INVENTION

There is now provided a pharmaceutical composition for application to an area of skin of a subject for local and/or systemic treatment of a COX-2 mediated disorder. The composition comprises a backing sheet that is flexibly conformable to the area of skin, the backing sheet having opposing surfaces that are respectively distal and proximal to the skin when applied; and a coating on the proximal surface of the backing sheet. The coating comprises (a) an adhesive and (b) a water-soluble active agent comprising a selective COX-2 inhibitory drug or prodrug or salt thereof, the active agent being in a therapeutically effective total amount and dispersed in a matrix that comprises zero to less than an active agent solubilizing effective amount in total of one or more solvents other than the adhesive.

When one or more solvents other than the adhesive, for example water or a polyhydric alcohol such as polyethylene glycol or propylene glycol, are defined herein as present in the matrix at “less than an active agent solubilizing effective amount in total”, it will be understood that the amount of such solvents is insufficient to dissolve all of the active agent present in the coating. The active agent can be dispersed in solid particulate form in the matrix. Alternatively, where the matrix is formed predominantly by the adhesive, the active agent can optionally be wholly or partly molecularly dispersed, i.e., in solid solution, in such a matrix.

The present compositions are not limited by any process used to prepare them. In an illustrative process, the active agent is dissolved in a solvent or mixture of solvents, for example ethanol and water, prior to mixing with the adhesive. However, typically the solvent or mixture of solvents is later removed by heating, thus the final composition, in accordance with the present invention, contains no such solvents or has less than an active agent solubilizing effective amount in total of such solvents.

In a preferred embodiment the coating comprises a layer having the active agent dispersed in a matrix that comprises the adhesive. Alternatively, the coating can comprise two layers: a reservoir layer that comprises the active agent adjacent to the backing sheet, and an adhesive layer that is proximal to the skin when applied. Optionally in such a coating, a membrane that permits passage of the active agent is present between the reservoir layer and the adhesive layer.

In preferred compositions, the coating further comprises one or more skin permeation enhancers.

Preferably a peelable release liner is also provided. This liner, prior to use, is adjacent to the layer that contains the adhesive, and is removed prior to application of the composition to the skin.

There is further provided a method of local treatment of pain and/or inflammation at a site thereof in a subject, the method comprising applying a pharmaceutical composition as provided herein to a skin surface of the subject, preferably at a locus overlying or adjacent to the site of pain and/or inflammation, and leaving the composition in place for a time period effective to permit delivery of a locally therapeutic amount of the active agent.

There is still further provided a method of systemic treatment of a subject having a COX-2 mediated disorder, the method comprising applying a pharmaceutical composition as provided herein to a skin surface of the subject, and leaving the composition in place for a time period effective to permit transdermal delivery of a therapeutic amount of the active agent.

### BRIEF DESCRIPTION OF THE DRAWINGS

FIG. 1 is a schematic drawing in section, not to scale, of a composition of a first embodiment of the invention.

FIG. 2 is a schematic drawing in section, not to scale, of a composition of a second embodiment of the invention.

### DETAILED DESCRIPTION OF THE INVENTION

A pharmaceutical composition of the invention is described herein as an “adhesive coated sheet”, a generic term which will be understood to embrace patches, tapes, poultices, pads, plasters, cataplasms and dressings that are adhesive to skin. The components of the adhesive coated sheet are described herein with reference to a skin surface to which the composition is to be applied. As applied to a layer or surface herein, the term “proximal” means toward the skin surface and the term “distal” means away from the skin surface, when the composition is correctly applied.

The most distal layer of the composition is a backing sheet that is flexibly conformable to the skin surface. Any suitable material can be used for the backing sheet, but typically a polymer film, e.g., one comprising one or more of polyethylene, polyvinyl chloride, ethyl vinyl acetate, polyurethane and polyester, or a woven or nonwoven fabric, optionally having a polymer film laminated thereon, is used. The backing sheet can be airtight and/or waterproof, providing a substantially occlusive dressing. Alternatively, a backing sheet can be used having pores or other means for circulation of air to the treated skin area. A presently preferred backing sheet is an ethyl vinyl acetate film having a thickness of about 20 to about 100 μm, for example Mediflex® 1200 of Mylan Technologies, Inc.

A coating is present on the proximal surface of the backing sheet. As indicated above, the coating comprises (a) an adhesive and (b) an active agent comprising valdecoxib or a prodrug thereof or a salt thereof, the active agent being in a therapeutically effective total amount and dispersed in a matrix that comprises zero to less than an active agent solubilizing effective amount in total of one or more solvents other than the adhesive.

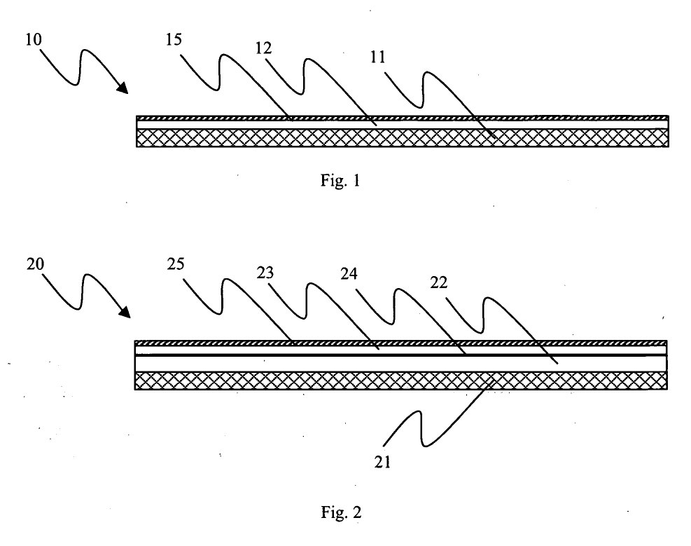

In a first embodiment, the active agent is dispersed in a matrix that comprises the adhesive and, optionally, other excipients. As shown in FIG. 1, a composition 10 of this first embodiment comprises a distal backing sheet 11 having on its proximal surface a coating layer 12 wherein the active ingredient is dispersed in an adhesive matrix. On the proximal side of the coating layer 12 is an optional peelable release liner 15 that can be removed to expose the coating layer 12 prior to application to a skin surface.

In a second embodiment, the active agent is dispersed in a solid or semi-solid matrix, for example a gel, in a reservoir layer adjacent to the backing sheet, and the adhesive is present in a distinct layer proximal to the reservoir layer, optionally with a membrane that permits passage of the active agent between these layers. As shown in FIG. 2, a composition 20 of this second embodiment comprises a distal backing sheet 21 having on its proximal surface a reservoir layer 22 wherein the active ingredient is dispersed in a solid or semi-solid matrix. On the proximal side of the reservoir layer 22 is an adhesive layer 23, optionally separated from the reservoir layer 22 by a membrane 24. On the proximal side of the adhesive layer 23 is an optional peelable release liner 25 that can be removed to expose the adhesive layer 23 prior to application to a skin surface.

Preferably in either of the above embodiments, a release liner is provided. This liner can be made of any suitable material that does not adhere to the adhesive-containing layer, or laminated with such a material, so that the liner is readily peelable without detaching a significant amount of that layer from the composition. Typical release liners are polyester, polyethylene, polypropylene, PET (polyethylene terephthalate) or polyurethane films laminated with a silicone or fluoropolymer easy-release coating. A presently preferred release liner is a silicone-laminated polyester, PET or polyurethane film having a thickness of about 50 to about 250 μm, for example Mediflex® 2228 of Mylan Technologies, Inc.

The release liner provides some protection for the coating during transport and storage of the composition, but typically the composition is additionally protected by individual packaging, for example a polyethylene wrap. The composition is preferably maintained in sterile condition until the packaging is opened.

The active agent comprises at least one compound selected from selective COX-2 inhibitory drugs and prodrugs and salts thereof having a solubility in water at 25° C. of at least about 10 mg/ml, preferably at least about 50 mg/ml and more preferably at least about 100 mg/ml. A “selective COX-2 inhibitory drug” herein is one having a COX-1/COX-2 IC₅₀ ratio, as measured in vivo or in vitro, of at least about 10, preferably at least about 20, more preferably at least about 50 and most preferably at least about 100.

In one embodiment the active agent comprises a water-soluble compound of formula (IX)

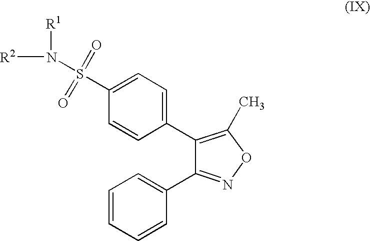

where R¹ and R² are independently hydrogen or a group that is metabolically replaceable, by hydrogen; or a pharmaceutically acceptable salt of such a compound. Preferably R¹ is hydrogen or a lower alkyl, hydroxyalkyl or acyl group and R² is hydrogen or a lower alkyl, hydroxyalkyl or acyl group or a group R³—CO— where R³ is hydrogen or a lower alkyl, lower alkoxy, lower carboxyalkyl, lower alkoxyalkyl, lower alkoxycarbonylalkyl, lower aminoalklyl, lower alkylcarbonylaminoalkyl, lower alkoxycarbonylaminoalkyl, phenyl or lower alkoxycarbonyl group, more preferably hydrogen or a C₁₋₅ alkyl, alkoxy. carboxyalkyl, alkoxyalkyl, aminoalkyl, alkoxycarbonyl or phenyl group.

Especially preferred are compounds of formula (IX) where R¹ is hydrogen and R² is ethoxycarbonyl (i.e., parecoxib), and salts of parecoxib having formula (X):

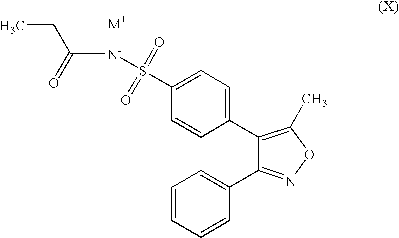

where M⁺ is a pharmaceutically acceptable cation. Such salts illustratively include base addition salts having inorganic cations such as alkali metal and alkaline earth metal cations, for example aluminum, calcium, lithium, magnesium, potassium, sodium and zinc, or organic cations prepared from amines such as tromethamine, diethylamine, N,N′-dibenzylethylenediamine, chloroprocaine, choline, diethanolamine, ethylenediamine, meglumine, procaine and the like. Preferred salts of parecoxib are alkali metal salts, most preferably the sodium salt, hereinafter referred to as parecoxib sodium.

In another embodiment the active agent comprises a water-soluble compound of formula (XI)

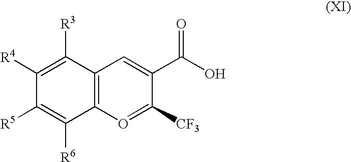

where R³ is hydrogen, halogen, or a lower alkyl, lower hydroxyalkyl or alkoxy group; R⁴ is hydrogen, halogen or a lower alkyl, lower alkylthio, lower haloalkyl, amino, aminosulfonyl, lower alkylsulfonyl, lower alkylsulfinyl, lower alkoxyalkyl, lower alkylcarbonyl, formyl, cyano, lower haloalkylthio, substituted or unsubstituted phenylcarbonyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5- or 6-membered heteroaryl, lower hydroxyalkyl, substituted or unsubstituted phenyl or 5- or 6-membered nitrogen-containing heterocyclosulfonyl group; R⁵ is hydrogen or halogen or a lower alkyl, lower haloalkyl, lower alkoxy or phenyl group; and R⁶ is hydrogen, halogen or a cyano, hydroxyiminomethyl, lower hydroxyalkyl, lower alkynyl, phenylalkynyl, lower alkyl, lower alkoxy, formyl or phenyl group; or a salt thereof.

Especially preferred are compounds of formula (XI) where R³ and R⁵ are each hydrogen and R⁴ and R⁶ are each chlorine, i.e., the compound (S)-6,8-dichloro-2-(trifluoromethyl)-2H-1-benzopyran-3-carboxylic acid (VI), or where R³ and R⁶ are each hydrogen, R⁴ is chlorine and R⁵ is a tert-butyl group, i.e., the compound (S)-6-chloro-7-(1,1-dimethylethyl)-2-(trifluoromethyl)-2H-1-benzopyran-3-carboxylic acid (VII), and pharmaceutically acceptable salts thereof

Parecoxib and its salts used in compositions of the invention can be prepared by any known process, for example in the manner set forth in above-cited U.S. Pat. No. 5,932,598. Compounds of formula (XI) and salts thereof used in compositions of the invention can be prepared by any known process, for example in the manner set forth in above-cited U.S. Pat. No. 6,034,256.

The active agent is present in an amount and at a concentration sufficient to provide therapeutic efficacy when the composition is applied to the skin and remains in contact therewith for a period of up to about 7 days, preferably up to about 1 day. What constitutes a therapeutically effective amount or concentration depends upon the particular active agent used, the permeability of the skin, the nature of the disorder to be treated, whether local or systemic delivery is required, and other factors.

Typically in the case of parecoxib or parecoxib sodium, a concentration in the composition, excluding the backing sheet, of about 0.1% to about 50%, more typically about 0.5% to about 25%, for example about 1% to about 10%, by weight is suitable. The amount of parecoxib or parecoxib sodium per unit area of the composition is typically about 10 to about 5000 μg/cm², more typically about 50 to about 2500 μg/cm², for example about 100 to about 1000 μg/cm². Illustratively, a 10 cm×10 cm (100 cm²) patch containing 200 μg active agent per cm² is equivalent to a 20 mg dose ofthe active agent, although only a fraction of the applied dose may be transported into and/or through the skin. For example, this illustrative patch may deliver the active agent at a permeation rate of 20 μg/cm².day for 1 day, equivalent to a total delivery of 2 mg of the active agent, or an efficiency of delivery of 2/20, i.e., 10%. Greater and lesser efficiencies of delivery are also within the scope contemplated herein.

The active agent is dispersed in a matrix that comprises zero to less than an active agent solubilizing amount in total of one or more solvents other than the adhesive. In a preferred embodiment, the matrix consists predominantly (more than about 50% by weight) of the adhesive.

Without being bound by theory, it is believed that having the active agent fully solubilized in a solvent system other than the adhesive would counteract to an undesirable degree the tendency of the active agent to penetrate into the skin and underlying tissues. By contrast, the present composition, being substantially free of such a solvent system, is believed to have greater driving force for transfer of the active agent into or through the skin than a composition as contemplated for example in above-cited U.S. Pat. No. 5,932,598 that includes the active agent “in a suitable solvent system with an adhesive system, such as an acrylic emulsion”.

Preferably the composition exhibits a skin permeation rate of not less than about 1, more preferably not less than about 3 and most preferably not less than about 10 μg/cm².day.

When a skin permeation rate or range of such rates is indicated herein, it will be understood to mean a rate as determined by a standard test, illustratively a standard test using human cadaver skin.

As an example of such a test, a Franz diffusion cell can be used having a cadaver skin membrane of suitable area, e.g., a disk of diameter 25 mm, and a suitable receptor fluid, for example 1% polysorbate 80 solution or a 6% polyethylene glycol (20) oleyl ether (oleth-20) solution. The receptor compartment of the Franz diffusion cell is filled with the receptor fluid and the diffusion cell is maintained at a suitable temperature, preferably a temperature approximating living human skin temperature. A receptor fluid temperature of 32° C. has been found suitable. The membrane is oriented so that its internal surface, i.e., the surface opposite the epidermal surface, is placed in contact with the receptor fluid. Air bubbles are removed from the receptor fluid, which is then allowed to equilibrate with the membrane for a suitable period, typically about 30 minutes. The epidermal surface is dried and a test sample, for example a 10 mm disk, of a composition, with any release layer having been removed, is placed with its adhesive coating in contact with the epidermal surface, and left in place for a desired period, for example 24 hours. It is important to ensure good integrity of contact between the sample and the epidermis. At intervals during this period, and/or at the end of this period, concentration of the active agent is determined in the receptor fluid by a suitable analytical method, e.g., high performance liquid chromatography (HPLC). This concentration is a measure of the amount of the active agent that has permeated the skin membrane during the period of the test, and can be used to calculate a skin permeation rate of active agent in units such as μg/cm².day or μg/cm².hour.

It will be understood that skin membranes exhibit significant variation in permeability, depending on source. Absolute permeation rates through such membranes are therefore less meaningful than permeation rates normalized for permeability of the test membrane used, based on data obtained with a reference composition. Suitable reference compositions are a solution of the active agent in 70% aqueous ethanol or an aqueous suspension, and can be evaluated in Franz cells or side-by-side diffusion cells.

The adhesive generally comprises one or more macromolecular substances. Examples include gelatin, agar, alginic acid, mannan, carboxymethylcellulose, methylcellulose, polyvinyl alcohol, natural rubber, polyisoprene, polybutadiene, polyisobutylene (PIB), styrene-isoprene-styrene (SIS) block copolymers, polyacrylic esters, polymethacrylic esters, acrylic ester-methacrylic ester copolymers, acrylic acid-acrylic ester-vinyl acetate copolymers and petroleum resins. Silicone-based adhesives are another option.

When a natural rubber is used as the base for an adhesive, an illustrative adhesive composition comprises about 30% to about 70% by weight of natural rubber, about 30% to about 60% by weight of a tackifier resin, not more than about 20% by weight of a plasticizer or softening agent and about 0.01% to about 2% of an antioxidant. When the adhesive is based on an SIS block copolymer, an illustrative adhesive composition comprises about 20% to about 50% by weight of the copolymer, about 25% to about 60% by weight of a tackifier resin, about 5% to about 20% by weight of a liquid rubber and about 0.01% to about 2% by weight of an antioxidant.

Suitable tackifier resins illustratively include alicyclic saturated hydrocarbon petroleum resins, rosin, rosin glycerol ester, hydrogenated rosin, hydrogenated rosin glycerol ester, hydrogenated rosin pentaerythritol ester, cumaroneindene resins, polyterpenes, terpene-phenolic resins, cycloaliphatic hydrocarbon resins, alkyl aromatic hydrocarbon resins, hydrocarbon resins, aromatic hydrocarbon resins and phenolic resins. Suitable antioxidants illustratively include dibutylhydroxytoluene (BHT). Suitable plasticizers or softening agents illustratively include liquid paraffm and petrolatum.

Optionally, a metal sequestering agent can be incorporated into the adhesive composition. Suitable sequestering agents include, among others, ethylene diamine tetraacetic acid (EDTA), potassium polyphosphate, sodium polyphosphate, potassium metaphosphate, sodium metaphosphate, dimethylglyoxime, 8-hydroxyquinoline, nitrilotriacetic acid, dihydroxyethylglycine, gluconic acid, citric acid and tartaric acid. These are illustratively used in an amount of about 0.01% to about 2% by weight.

Presently preferred adhesives, generally provided in solution in one or more solvents, are PIB based adhesives, for example Duro-Tak® 87-6173 of National Starch; acrylate based adhesives, for example Duro-Tak® 387-2052, 387-2353 or 387-2516 of National Starch; and silicone based adhesives, for example Bio-PSA® 7-4201 of Dow Corning. The selection of an optimum adhesive system for use in a particular composition of the invention can, in light of the disclosure herein, be made by routine testing, but it will generally be found that for best skin flux of valdecoxib, an acrylic based adhesive system should be selected, while for best skin flux of parecoxib, especially when applied as parecoxib sodium, a silicone based adhesive system is preferable.

It is preferred to include at least one skin permeation enhancer in the composition.

In one embodiment, the at least one skin permeation enhancer is selected from terpenes, terpenoids, fatty alcohols and derivatives thereof. Examples include oleyl alcohol, thymol, menthol, carvone, carveol, citral, dihydrocarveol, dihydrocarvone, neomenthol, isopulegol, 4-terpinenol, menthone, pulegol, camphor, geraniol, α-terpineol, linalool, carvacrol, trans-anethole, isomers thereof and racemic mixtures thereof. Optionally more than one such permeation enhancer, for example a fatty alcohol and a terpene or terpenoid, can be present. Thus, in an illustrative embodiment, a composition of the invention comprises as penetration enhancers oleyl alcohol and thymol.

Fatty acids such as oleic acid and their alkyl and glyceryl esters such as isopropyl laurate, isopropyl myristate, methyl oleate, glyceryl monolaurate, glyceryl monooleate, glyceryl monostearate, glyceryl dilaurate, glyceryl dioleate, etc. also can be used as skin permeation enhancers. Of this group, glyceryl monolaurate is especially preferred. Fatty acid esters of glycolic acid and its salts, for example as disclosed in International Patent Publication No. WO 98/18416, incorporated herein by reference, are also useful skin permeation enhancers. Examples of such esters include lauroyl glycolate, caproyl glycolate, cocoyl glycolate, isostearoyl glycolate, sodium lauroyl glycolate, tromethamine lauroyl glycolate, etc. Also useful as skin permeation enhancers are lactate esters of fatty alcohols, for example lauryl lactate, myristyl lactate, oleyl lactate, etc.

Other skin permeation enhancers include hexahydro-1-dodecyl-2H-azepin-2-one (laurocapram, Azone™) and derivatives thereof, dimethylsulfoxide (DMSO), n-decyl methylsulfoxide, salicylic acid and alkyl esters thereof, e.g., methyl salicylate, N,N-dimethylacetamide, dimethylformamide, N,N-dimethyltoluamide, 2-pyrrolidinone and N-alkyl derivatives thereof, e.g., NMP and N-octyl-2-pyrrolidinone, 2-nonyl-1,3-dioxolane, eucalyptol and sorbitan esters.

Other ingredients of the composition can include one or more excipients selected from thickening agents, surfactants, emulsifiers, antioxidants, preservatives, stabilizers, colors and fragrances. A skin irritation reducing agent, such as vitamin E, glycyrrhetic acid or diphenhydramine, can also be present.

Illustratively a composition of the invention has a coating layer that comprises amounts of various ingredients as follows (all percentages by weight):

| | |
|---|---|
| active agent | 1-10% |
| skin permeation enhancer(s) | 2-20% |
| adhesive(s) | 70-97% |

Certain compounds listed above as permeation enhancers can function as topical analgesics in their own right. For example, methyl salicylate, menthol or a combination thereof can provide complementary analgesia when included in a composition of the present invention. In particular, such compounds can provide early-onset, short-term analgesia that complements the longer-term, sustained analgesic and anti-inflammatory effects of the active agent. In compositions of the invention comprising methyl salicylate and menthol, suitable amounts are 5-30% by weight of methyl salicylate and 2-20% by weight of menthol. Amounts outside these ranges can also be useful in particular situations.

Coated sheet compositions of the invention can be prepared by any known process. Two illustrative processes are described herein as a “solvent process” and a “hot melt process”.

According to the solvent process, the active agent is first dissolved in a suitable solvent that can readily be removed later by heating. Depending on the active: agent, the solvent can be aqueous, organic or a mixture thereof. Suitable examples include ethanol, ethanol/water mixture, ethyl acetate, isopropanol, toluene and heptane. Optionally, one or more excipient ingredients other than the adhesive, including for example one or more skin permeation enhancers, are added to the resulting solution, which is mixed thoroughly, with agitation and/or sonication if necessary, to form a premix. The adhesive is provided in solution in a suitable solvent that can readily be removed later by heating. The adhesive solution is added to the premix with thorough mixing to ensure a homogeneous mixture. It is usually desirable to conduct this mixing in a way that minimizes air entrapment, or to remove air from the mixture before proceeding to the next step. The mixture is then coated on a suitable release liner at a desired thickness. The resulting coated liner is dried to remove most, preferably substantially all, of the solvents introduced in the premix and the adhesive solution. Drying can take place under any set of conditions effective for such drying, but typically a short drying period at ambient temperature is followed by a period of drying at elevated temperature. Drying temperatures should be selected to be sufficiently high to drive off the solvents but not so high as to cause significant degradation of the active agent or other ingredients. After drying, a suitable backing sheet is placed over the coating on the liner and is pressed to ensure good contact between the coating and the backing sheet. The resulting coated sheet composition can be cut to any desired size and packaged in any suitable packaging, for example a polyethylene or metallic foil pouch.

According to the hot melt process, a pressure-sensitive adhesive composition is first provided. Typically such a composition comprises a thermoplastic polymer system such as natural rubber or a styrenic block copolymer (e.g., SIS), a tackifier resin, a plasticizer and an antioxidant. The adhesive composition is heated with mixing, at a temperature sufficient to melt the adhesive but not so high as to cause significant degradation of the active agent. The active agent is added in powder or molten form to the resulting melted adhesive, with thorough mixing to provide a coating composition, which is then coated on a suitable release liner at a desired thickness. A suitable backing sheet is placed over the coating on the liner and is pressed to ensure good contact between the coating and the backing sheet. The resulting coated sheet composition can be cut and packaged as in the solvent process.

The composition can be designed so that the drug penetrates the skin to deliver a therapeutically effective amount of the drug to a target site such as epidermal, dermal, subcutaneous, muscular and articular organs and tissues while maintaining systemic levels of the drug not greatly in excess of a minimum therapeutically effective level. Thus the present composition can be used to effect targeted delivery of a water-soluble selective COX-2 inhibitory drug or prodrug to an external or internal site of pain and/or inflammation in a subject. According to a first therapeutic method of the invention, a composition as provided herein is topically administered to a skin surface of the subject, preferably at a locus overlying or adjacent to the site of pain and/or inflammation.

Compositions as provided herein can alternatively be used to effect systemic treatment of a subject having a COX-2 mediated disorder. According to a second therapeutic method of the invention, a composition as provided herein is administered transdermally, preferably by contacting the composition with an area of skin of the subject not greater than about 400 cm².

Therapeutic methods and compositions of the invention are useful in treatment and prevention of a very wide range of disorders mediated by COX-2, including but not restricted to disorders characterized by inflammation, pain and/or fever. Such compositions are especially useful as anti-inflammatory agents, such as in treatment of arthritis, with the additional benefit of having significantly less harmful side effects, especially when systemically administered, than compositions of conventional NSAIDs that lack selectivity for COX-2 over COX-1. Thus compositions of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendinitis, bursitis, allergic neuritis, cytomegalovirus infectivity, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, scleroderma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic diseases, such as retinitis, conjunctivitis, retinopathies, uveitis, ocular photophobia, and of acute injury to the eye tissue.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are used in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemangiomas, angiofibroma of the nasopharynx and a vascular necrosis of bone; and disorders of the female reproductive system such as endometriosis.

Such compositions are useful in the treatment of pre-cancerous diseases, such as actinic keratosis.

Such compositions are useful in prevention, treatment and inhibition of benign and malignant tumors and neoplasia including neoplasia in metastasis, for example in colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in patients at risk of FAP.

More particularly, the compositions can be used in treatment, prevention and inhibition of acral lentiginous melanoma, actinic keratoses, adenocarcinoma, adenoid cystic carcinoma, adenoma, adenosarcoma, adenosquamous carcinoma, astrocytic tumors, bartholin gland carcinoma, basal cell carcinoma, breast cancer, bronchial gland carcinoma, capillary hemangioma, carcinoids, carcinosarcoma, cavernous hemangioma, cholangiocarcinoma, chondrosarcoina, chorioid plexus papilloma or carcinoma, clear cell carcinoma, cutaneous T-cell lymphoma (mycosis fungoides), cystadenoma, displastic nevi, endodermal sinus tumor, endometrial hyperplasia, endometrial stromal sarcoma, endometrioid adenocarcinoma, ependymoma, epithelioid angiomatosis, Ewing's sarcoma, fibrolamellar sarcoma, focal nodular hyperplasia, gastrinoma, germ cell tumors, glioblastoma, glucagonoma, hemangioblastoma, hemangioendothelioma, hemangioma, hepatic adenoma, hepatic adenomatosis, hepatocellular carcinoma, insulinoma, intraepithelial neoplasia, interepithelial squamous cell neoplasia, invasive squamous cell carcinoma, Kaposi's sarcoma, large cell carcinoma, leiomyosarcoma, lentigo-maligna melanoma, malignant melanoma, malignant mesothelial tumors, medulloblastoma, medulloepithelioma, melanoma, meningioma, mesothelioma, mucoepidermoid carcinoma, neuroblastoma, neuroepithelial adenocarcinoma, nodular melanoma, oat cell carcinoma, oligodendroglioma, osteosarcoma, papillary serous adenocarcinoma, pineal tumors, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinoma, somatostatin-secreting tumor, squamous carcinoma, squamous cell carcinoma, submesothelial carcinoma, superficial spreading melanoma, undifferentiated carcinoma, uveal melanoma, verrucous carcinoma, vipoma, well differentiated carcinoma and Wilm's tumor.

Such compositions inhibit prostanoid-induced smooth muscle contraction by inhibiting synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Preferred uses for compositions of the invention are for treatment of rheumatoid arthritis and osteoarthritis, for pain management generally (particularly post-oral surgery pain, post-general surgery pain, post-orthopedic surgery pain, and acute flares of osteoarthritis), for prevention and treatment of headache and migraine, for treatment of Alzheimer's disease, and for colon cancer chemoprevention.

Topical application of a composition of the invention can be especially useful in treatment of any kind of dermal disorder having an inflammatory component, whether malignant, non-malignant or pre-malignant, including scar formation and ketosis, and also including burns and solar damage, for example sunburn, wrinkles, etc. Such compositions can be used to treat inflammation resulting from a variety of skin injuries including without limitation those caused by viral diseases including herpes infections (e.g., cold sores, genital herpes), shingles and chicken pox. Other lesions or injuries to the skin that can be treated with such compositions include pressure sores (decubitus ulcers), hyperproliferative activity in the epidermis, miliria, psoriasis, eczema, acne, dermatitis, itching, warts and rosacea. Such compositions can also facilitate healing processes after surgical procedures, including cosmetic procedures such as chemical peels, laser treatment, dermabrasion, face lifts, eyelid surgery, etc.

Besides being useful for human treatment, compositions of the invention are also useful for veterinary treatment of companion animals, exotic animals, farm animals, and the like, particularly mammals including rodents. More particularly, compositions of the invention are useful for veterinary treatment of COX-2 mediated disorders in horses, dogs and cats.

The present compositions can be used in combination therapies with opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise use of a composition of the invention with one or more compounds selected from aceclofenac, acemetacin, ε-acetamidocaproic acid, acetaminophen, acetaminosalol, acetanilide, acetylsalicylsalicylic acid, S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, aspirin, balsalazide, bendazac, benorylate, benoxaprofen, benzpiperylon, benzydamine, benzylmorphine, berberine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butorphanol, calcium acetylsalicylate, carbamazepine, carbiphene, carprofen, carsalam, chlorobutanol, chlorthenoxazin, cholline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, dipyrocetyl, dipyrone, ditazol, droxicam, emorfazone, enfenamic acid, epirizole, eptazocine, etanercept, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, infliximab, interleukin-10, isofezolac, isoladol, isomethadone, isonixin, isoxepac, isoxicam, ketobemidone, ketoprofen, ketorolac, p-lactophenetide, lefetamnine, levorphanol, lexipafant, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone, methotrimeprazine, metiazinic acid, metofoline, metopon, mofebutazone, mofezolac, morazone, morphine, morphine hydrochloride, morphine sulfate, morpholine salicylate, myrophine,, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl, acetylsalicylate, phenylbutazone, phenyl salicylate, phenyramidol, piketoprofen, piminodine, pipebuzone, piperylone, pirazolac, piritramide, piroxicam, pirprofen, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalate, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen, ziconotide and zomepirac (see The Merck Index, 13th Edition (2001), Therapeutic Category and Biological Activity Index, lists therein headed “Analgesic”, “Anti-inflammatory” and “Antipyretic”).

Particularly preferred combination therapies comprise use of a composition of the invention with an opioid compound, more particularly where the opioid compound is codeine, meperidine, morphine or a derivative thereof.

The compound to be administered in combination with the composition of the invention can be formulated separately therefrom, and administered by any suitable route, including orally, rectally, parenterally or topically to the skin or elsewhere. Alternatively, the compound to be administered in combination with the present composition can be coformulated therewith as a coated sheet composition.

In an embodiment of the invention, particularly where the COX-2 mediated condition is headache or migraine, the present composition is administered in combination therapy with a vasomodulator, preferably a xanthine derivative having vasomodulatory effect, more preferably an alkylxanthine compound.

Combination therapies wherein an alkylxanthine compound is co-administered with a composition as provided herein are embraced by the present embodiment of the invention whether or not the alkylxanthine is a vasomodulator and whether or not the therapeutic effectiveness of the combination is to any degree attributable to a vasomodulatory effect. The term “alkylxanthine” herein embraces xanthine derivatives having one or more C₁₋₄ alkyl, preferably methyl, substituents, and pharmaceutically acceptable salts of such xanthine derivatives. Dimethylxanthines and trimethylxanthines, including caffeine, theobromine and theophylline, are especially preferred. Most preferably, the alkylxanthine compound is caffeine.

The vasomodulator or alkylxanthine component of the combination therapy can be administered in any suitable dosage form by any suitable route, including orally, rectally, parenterally or topically to the skin or elsewhere. The vasomodulator or alkylxanthine can optionally be coformulated with the present composition in a single transdermal dosage form. Thus a transdermal composition of the invention optionally comprises both a water-soluble selective COX-2 inhibitory drug or prodrug and a vasomodulator or alkylxanthine such as caffeine, in total and relative amounts that are therapeutically effective.

### EXAMPLES

This invention will be more fully described by way of the following Examples but is not limited to these Examples. The term “parecoxib” is used in these Examples in the strict sense of parecoxib acid except where otherwise indicated; “parecoxib Na” means parecoxib sodium.

As a way of measuring the skin permeation properties of selective COX-2 inhibitory drugs or prodrugs as formulated in test patches, a Franz diffusion cell was provided utilizing a human cadaver skin membrane and a receptor fluid such as 1% polysorbate 80 (Tween™ 80) solution or 6% oleth-20 (Brij™ 98) solution. The receptor compartment of the Franz diffusion cell was filled with the receptor fluid and the diffusion cell was maintained at 32° C. Frozen skin was thawed at room temperature and a 2×2 cm square was cut out to provide a membrane. The surface of the membrane was dried with a cotton swab. A 10 mm disk (area 0.636 cm²) was punched out of a test patch and this disk was applied with its adhesive side in contact with the membrane. To ensure good contact between the test patch and the membrane, a 2 kg weight was run over the patch three times and a piece of stiff, clear plastic was placed upon the patch on the membrane. The membrane with the test patch mounted thereon was then placed on the receptor compartment, covered and fastened with a clamp. Air bubbles were removed from the receptor fluid, which was allowed to equilibrate for 30 minutes. The amount of drug that had permeated through the membrane by various times in a 24 to 48 hour period was determined by HPLC analysis of the receptor fluid. Each test was conducted in several replicates.

### Example 1

Patch formulations of parecoxib sodium were prepared as follows. Parecoxib sodium and the other ingredients shown in Table 1, with the exception of the Duro-Tak® adhesive, were weighed and dissolved in ethanol to provide an ethanol solution. The adhesive, provided at known solids content in solution, was weighed and mixed with the ethanol solution. Air was removed from the resulting mixture, which was then laminated on a release liner (Medirelease® 2228) with a drawdown device to form a coating of thickness 1-2 mm. The laminated solution was dried at room temperature for 5-10 minutes and then dried in an oven at 40-80° C. for 20-40 minutes to remove essentially all solvent. A backing sheet (Mediflex® 1200) was placed on the coated side of the liner, and it was pressed with a brayer. The resulting 10 cm×30 cm patches were stored in plastic bags.

TABLE 1
Composition of parecoxib sodium patch coatings

| | amount added (g)¹ | | | dry weight (%) | | |
|---|---|---|---|---|---|---|
| | Composition No.: | | | | | |
| Ingredient | 1-1 | 1-2 | 1-3 | 1-1 | 1-2 | 1-3 |
| Duro-Tak ® 387-2052², | 11.5 | 8.9 | | 86.7 | 83.6 | |
| 47.5% solids | | | | | | |
| Duro-Tak ® 87-6173³, | | | 7.5 | | | 89.3 |
| 65% solids | | | | | | |
| parecoxib Na | 0.124 | 0.093 | 0.087 | 1.97 | 1.84 | 1.78 |
| thymol | 0.065 | 0.049 | 0.046 | 1.04 | 0.97 | 0.94 |
| oleyl alcohol | 0.132 | 0.099 | 0.092 | 2.10 | 1.96 | 1.89 |
| lauryl lactate | 0.129 | 0.097 | 0.091 | 2.05 | 1.92 | 1.86 |
| myristyl lactate | 0.127 | 0.095 | 0.089 | 2.01 | 1.88 | 1.82 |
| glyceryl dilaurate | 0.129 | 0.097 | 0.091 | 2.05 | 1.92 | 1.86 |
| propylene glycol | 0.132 | 0.099 | 0.092 | 2.10 | 1.96 | 1.89 |
| sesame oil | | 0.20 | | | 4.0 | |

¹adhesive amount on wet weight basis
²acrylate adhesive
³PIB adhesive

### Example 2

The patches of Example 1 were tested for skin permeation properties, using a skin of low permeability. Skin flux data are shown in Table 2. Time course of skin permeation is shown graphically in FIG. 3. Composition 1-3, containing a PIB adhesive, exhibited somewhat lower skin permeation than compositions 1-1 and 1-2, containing an acrylate adhesive. It will be noted that composition 1-3 had a lower concentration of parecoxib sodium than the other two compositions of this example.

TABLE 2
Skin flux of parecoxib from patches of Example 1

| Composition | Skin flux (μg/cm² · day) |
|---|---|
| 1-1 | 15.8 ± 4.1 |
| 1-2 | 17.7 ± 4.0 |
| 1-3 | 10.4 ± 3.0 |

When data for patch compositions 1-1 to 1-3 are normalized for the permeability of the skin source used in this study, these patches are seen to be capable of delivering a parecoxib skin flux of 50-100 μg/cm².day, equivalent, for a patch size of 100 cm², to a therapeutically useful rate of transdermal delivery of parecoxib of 5-10 mg/day.

As shown in FIG. 3, a continuous and steady delivery profile was observed over a period of 4 days, indicating that sustained therapeutically effective drug concentrations in plasma are achievable for several days, following application of a transdermal patch similar to those of compositions 1-1 to 1-3.

### Example 3

A patch formulation (composition 3-1) of parecoxib sodium was prepared as follows. Parecoxib sodium (0.99 g) and the other ingredients shown in Table 3, with the exception of the Duro-Tak® adhesive, (thymol 0.99 g, oleyl alcohol 1.00 g, lauryl lactate 1.01 g, PVP 1.07 g) were weighed and dissolved in a mixture of 7.57 g ethanol and 1.12 g water to provide a first mixture of total weight 13.74 g. The adhesive, provided at 36.5% solids content in solution, was weighed and 4.5 g of the first mixture added thereto to provide a second mixture. Mixing with slow rotation to avoid air entrapment continued for 2 hours. The resulting mixture was then laminated on a release liner (Medirelease® 2228) with a drawdown device to form a coating. The laminated solution was dried at room temperature for 15 minutes and then dried in an oven at 45° C. for 30 minutes to remove essentially all solvent. A backing sheet (Mediflex® 1200) was placed on the coated side of the liner, and it was pressed with a brayer. The resulting 10 cm×30 cm patches were stored in plastic bags.

Patch formulations (compositions 3-2 and 3-3) of parecoxib acid were prepared as follows. Parecoxib and the other ingredients shown in Table 3, with the exception of the PVP and the Duro-Tak® adhesive, were weighed together with 8.5 g ethanol and 0.5 g water to provide a first mixture of total weight 14.7 g. The first mixture was stirred and sonicated for 2 hours. It was noted that the parecoxib did not completely dissolve. The adhesive, provided at known solids content in solution, was weighed and a weighed amount of the first mixture (1.5 g in composition 3-2; 2.0 g in composition 3-3) added thereto to provide a second mixture. In composition 3-2 only, 1.1 g PVP was also added. Mixing with slow rotation to avoid air entrapment continued for 1 hour. This mixing appeared to result in complete dissolution of the parecoxib. The resulting mixture was then laminated on a release liner (Medirelease® 2228) with a drawdown device to form a coating. The laminated solution was dried in an oven at 45° C. for 2 hours to remove essentially all solvent. A backing sheet (Mediflex® 1200) was placed on the coated side of the liner, and it was pressed with a brayer. The resulting 10 cm×30 cm patches were stored in plastic bags.

TABLE 3
Composition of patch coatings

| | amount added (g)¹ | | | dry weight (%) | | |
|---|---|---|---|---|---|---|
| | Composition No.: | | | | | |
| Ingredient | 3-1 | 3-2 | 3-3 | 3-1 | 3-2 | 3-3 |
| Duro-Tak ® 387-2052², | | 8.8 | 11.5 | | 71.3 | 87.6 |
| 47.5% solids | | | | | | |
| Duro-Tak ® 387-2353³, | 11.5 | | | 71.8 | | |
| 36.5% solids | | | | | | |
| parecoxib Na | 0.32 | | | 5.5 | | |
| parecoxib | | 0.11 | 0.15 | | 1.9 | 2.4 |
| thymol | 0.32 | 0.10 | 0.14 | 5.5 | 1.8 | 2.2 |
| oleyl alcohol | 0.33 | 0.10 | 0.13 | 5.6 | 1.8 | 2.2 |
| lauryl lactate | 0.33 | 0.11 | 0.15 | 5.6 | 1.9 | 2.4 |
| PEG 400 | | 0.15 | 0.20 | | 2.6 | 3.2 |
| PVP, micronized⁴ | 0.35 | 1.10 | | 6.0 | 18.8 | |

¹adhesive amount on wet weight basis
^{2,3}acrylate adhesives
⁴Kollidon ® CL

### Example 4

The patches of Example 3 were tested for skin permeation properties, using a skin of low permeability. Skin flux data are shown in Table 4. Composition 1-3, containing a PIB adhesive, exhibited somewhat lower skin permeation than compositions 1-1 and 1-2, containing an acrylate adhesive. It will be noted that composition 1-3 had a lower concentration of parecoxib sodium than the other two compositions of this example.

TABLE 4
Skin flux of parecoxib from patches of Example 3

| Composition | Skin flux (μg/cm² · day) |
|---|---|
| 3-1 | 2.52 ± 2.50 |
| 3-2 | 1.51 ± 0.56 |
| 3-3 | 1.55 ± 0.50 |

### Example 5

Patch formulations compositions 5-1 to 5-24 were prepared having as active agent celecoxib, valdecoxib, parecoxib or parecoxib sodium. In general the procedure was as follows. The adhesive, provided at known solids content in solution, the active agent and, if required, other ingredients, were weighed and mixed together. Air was removed from the resulting mixture, which was then laminated on a release liner using a laboratory-scale knife-coater at a thickness of 0.45 mm. The composition was dried at room temperature for 5 minutes and then in an oven at 60° C. for 20 minutes. A PET backing sheet (Bertek® 92GA2600) was placed on the coated side of the liner, and it was pressed with a brayer. The resulting patches were stored in plastic bags.

The release liner was Medirelease® 2226 except where a silicone based adhesive was used, in which case Scotchpak® 1022 was selected as the release liner.

In some of the compositions of this example, an enhancer mixture was included. The enhancer mixture consisted of five skin permeation enhancers in ethyl acetate solution, having the following composition:

| | |
|---|---|
| thymol | 5.9% |
| oleyl alcohol | 11.8% |
| lauryl lactate | 11.8% |
| myristyl lactate | 11.8% |
| glyceryl dilaurate | 29.4% |
| ethyl acetate | 29.4% |

In other compositions, a single skin permeation enhancer (glyceryl monolaurate, herein “GML”) was added.

Three different adhesives were used: (1) a polyisobutylene (PIB) based adhesive, Duro-Tak® 87-6173 of National Starch, (2) an acrylate based adhesive, Duro-Tak® 387-2516 of National Starch, and (3) a silicone based adhesive, Bio-PSA® 7-4201 of Dow Corning.

Compositions of the coatings used in compositions 5-1 to 5-24 are shown in Table 5.

TABLE 5
Composition (% dry weight) of patch coatings

| | active agent | | enhancer | | adhesive | |
|---|---|---|---|---|---|---|
| Composition No. | identity | % | identity | % | identity | % |
| 5-1 | celecoxib | 6.0 | | 0 | PIB | 94.0 |
| 5-2 | celecoxib | 6.0 | GML | 10.0 | PIB | 84.0 |
| 5-3 | valdecoxib | 6.0 | | 0 | PIB | 94.0 |
| 5-4 | valdecoxib | 6.0 | GML | 10.0 | PIB | 84.0 |
| 5-5 | valdecoxib | 5.7 | GML | 4.8 | PIB | 89.5 |
| 5-6 | valdecoxib | 6.0 | mixture | 10.0 | PIB | 84.0 |
| 5-7 | valdecoxib | 6.7 | | 0 | acrylate | 93.3 |
| 5-8 | valdecoxib | 6.0 | GML | 10.0 | acrylate | 84.0 |
| 5-9 | valdecoxib | 6.3 | GML | 5.3 | acrylate | 88.4 |
| 5-10 | valdecoxib | 6.0 | mixture | 10.0 | acrylate | 84.0 |
| 5-11 | valdecoxib | 6.7 | | 0 | silicone | 93.3 |
| 5-12 | valdecoxib | 6.0 | mixture | 10.0 | silicone | 84.0 |
| 5-13 | parecoxib Na | 6.0 | | 0 | PIB | 94.0 |
| 5-14 | parecoxib Na | 6.0 | GML | 10.0 | PIB | 84.0 |
| 5-15 | parecoxib Na | 5.7 | GML | 4.8 | PIB | 89.5 |
| 5-16 | parecoxib Na | 6.0 | mixture | 10.0 | PIB | 84.0 |
| 5-17 | parecoxib Na | 6.7 | | 0 | acrylate | 93.3 |
| 5-18 | parecoxib Na | 6.0 | GML | 10.0 | acrylate | 84.0 |
| 5-19 | parecoxib Na | 6.3 | GML | 5.3 | acrylate | 88.4 |
| 5-20 | parecoxib Na | 6.0 | mixture | 10.0 | acrylate | 84.0 |
| 5-21 | parecoxib Na | 6.7 | | 0 | silicone | 93.3 |
| 5-22 | parecoxib Na | 6.0 | mixture | 10.0 | silicone | 84.0 |
| 5-23 | parecoxib | 6.0 | | 0 | PIB | 94.0 |
| 5-24 | parecoxib | 6.0 | GML | 10.0 | PIB | 84.0 |

### Example 6

Compositions 5-7 to 5-24 were tested for skin permeation. Skin flux data, normalized to correct for differences in permeability of different skin sources, are shown in Table 6.

TABLE 6
Skin flux of active agent from patches of Example 5

| Composition | Description | Skin flux (μg/cm² · day) |
|---|---|---|
| 5-7 | valdecoxib, acrylate adhesive | 18.7 ± 11.4 |
| 5-8 | cf. 5-7 with 10% GML | 22.1 ± 17.5 |
| 5-9 | cf. 5-7 with 5.3% GML | 35.6 ± 6.0 |
| 5-10 | cf. 5-7 with enhancer mixture | 24.1 ± 2.1 |
| 5-11 | valdecoxib, silicone adhesive | 5.9 ± 0.2 |
| 5-12 | cf. 5-11 with enhancer mixture | 9.2 ± 0.6 |
| 5-13 | parecoxib Na, PIB adhesive | 6.4 ± 0.5 |
| 5-14 | cf. 5-13 with 10% GML | 7.9 ± 1.5 |
| 5-15 | cf. 5-13 with 4.8% GML | 9.0 ± 0.6 |
| 5-16 | cf. 5-13 with enhancer mixture | ≦12.2 (n = 2) |
| 5-17 | parecoxib Na, acrylate adhesive | 7.6 ± 0.1 |
| 5-18 | cf. 5-17 with 10% GML | 8.9 ± 0.0 |
| 5-19 | cf. 5-17 with 5.3% GML | 10.9 ± 1.5 |
| 5-20 | cf. 5-17 with enhancer mixture | 12.1 ± 0.1 |
| 5-21 | parecoxib Na, silicone adhesive | 15.1 ± 2.5 |
| 5-22 | cf. 5-21 with enhancer mixture | 20.4 ± 11.5 |

The data in Table 6 indicate that:

(a) the selection of adhesive for best skin permeation appeared to depend on the active agent used (valdecoxib versus parecoxib sodium); and

(b) inclusion of enhancers (either a mixture of enhancers or glyceryl monolaurate alone) in a patch generally increased skin flux, regardless of active agent or adhesive.

### Example 7

Parecoxib sodium patch compositions 7-1 and 7-2 were prepared by a procedure similar to that described in Example 5. Each composition contained 5% parecoxib sodium, 5% enhancer and 90% acrylate adhesive (Duro-Tak® 385-2353) on a dry weight basis. The enhancer was glyceryl monolaurate (GML) in composition 7-1 and glyceryl monostearate (GMS) in composition 7-2.

Compositions 7-1 and 7-2 were tested for skin permeability. Skin flux data are shown in Table 7.

TABLE 7
Skin flux of active agent from patches of Example 7

| Composition | Description | Skin flux (μg/cm² · day) |
|---|---|---|
| 7-1 | parecoxib Na with 5% GML | 10.1 ± 2.7 |
| 7-2 | parecoxib Na with 5% GMS | 10.9 ± 0.8 |

### Example 8

Patch formulations compositions 8-1 to 8-26 were prepared having as active agent valdecoxib, parecoxib or parecoxib sodium. In general the procedure was as follows. Ethyl acetate in an amount of 6.6 to 8.1 g was weighed into a jar. Weighed amounts of the active agent and, if required, one or more enhancers, were added to the jar and mixed with sonication until a homogeneous mixture was obtained. An adhesive, provided at known solids content in solution, was added and mixed with a propeller mixer at speed setting 5 for 2 minutes. Air was removed from the resulting mixture, which was then laminated on a release liner (Scotchpak® 1022 of 3M except where otherwise noted below) using a laboratory-scale drawdown device at a thickness of 0.45 mm. The composition was dried at room temperature for 5 minutes and then in an oven at 60° C. for. 20 minutes. A backing sheet (CoTran® 9722 of 3M) was placed on the coated side of the liner, and it was pressed with a brayer. The resulting patches were stored in plastic bags.

Enhancers used were glyceryl monolaurate (GML), glyceryl monostearate (GMS) and lauryl lactate (LL).

Three different types of adhesive were used: (1) a polyisobutylene (PIB) based adhesive, Duro-Tak® 87-6173 of National Starch, (2) an acrylate based adhesive, Duro-Tak® 387-2052 of National Starch, and (3) a silicone based adhesive, Bio-PSA® 7-4302 of Dow Corning.

Compositions of the coatings used in compositions 8-1 to 8-26 are shown in Table 8.

TABLE 8
Composition (% dry weight) of patch coatings

| Composition | active agent | | enhancer | | adhesive | |
|---|---|---|---|---|---|---|
| No. | identity | % | identity | % | identity | % |
| 8-1 | valdecoxib | 6 | | 0 | PIB | 94 |
| 8-2 | valdecoxib | 6 | GML | 5 | PIB | 89 |
| 8-3 | valdecoxib | 6 | | 0 | acrylate | 94 |
| 8-4 | valdecoxib | 6 | GML | 5 | acrylate | 89 |
| 8-5 | valdecoxib | 6 | GMS | 5 | acrylate | 89 |
| 8-6 | valdecoxib | 6 | GML + | 5 + 5 | acrylate | 84 |
| | | | LL | | | |
| 8-7 | valdecoxib | 6 | | 0 | silicone | 94 |
| 8-8 | valdecoxib | 6 | GML | 5 | silicone | 89 |
| 8-9 | valdecoxib | 6 | GMS | 5 | silicone | 89 |
| 8-10 | valdecoxib | 6 | GML + | 5 + 5 | silicone | 84 |
| | | | LL | | | |
| 8-11 | parecoxib Na | 6 | | 0 | PIB | 94 |
| 8-12 | parecoxib Na | 6 | GML | 5 | PIB | 89 |
| 8-13 | parecoxib Na | 6 | | 0 | acrylate | 94 |
| 8-14 | parecoxib Na | 6 | GML | 5 | acrylate | 89 |
| 8-15 | parecoxib Na | 6 | GMS | 5 | acrylate | 89 |
| 8-16 | parecoxib Na | 6 | GML + | 5 + 5 | acrylate | 84 |
| | | | LL | | | |
| 8-17 | parecoxib Na | 6 | | 0 | silicone | 94 |
| 8-18 | parecoxib Na | 6 | GML | 5 | silicone | 89 |
| 8-19 | parecoxib Na | 6 | GMS | 5 | silicone | 89 |
| 8-20 | parecoxib Na | 6 | GML + | 5 + 5 | silicone | 84 |
| | | | LL | | | |
| 8-21 | parecoxib | 6 | | 0 | PIB | 94 |
| 8-22 | parecoxib | 6 | GML | 5 | PIB | 89 |
| 8-23 | parecoxib | 6 | | 0 | acrylate | 94 |
| 8-24 | parecoxib | 6 | GML | 5 | acrylate | 89 |
| 8-25 | parecoxib | 6 | | 0 | silicone | 94 |
| 8-26¹ | parecoxib | 6 | GML | 5 | silicone | 89 |

¹Medirelease ® 2500 release liner

Assay by HPLC showed that the amount of active agent present in the patches of Example 8 ranged from 149 to 799 μg/cm², with an average of 433 μg/cm².

### Example 9

Compositions 8-3 to 8-26 were tested for skin permeability. Skin flux data, normalized to correct for differences in permeability of different skin sources, are shown in Table 9.

TABLE 9
Skin flux of active agent from patches of Example 8

| Composition | Description | Skin flux (μg/cm² · day) |
|---|---|---|
| 8-3 | valdecoxib, acrylate adhesive | 2.09 ± 0.29 |
| 8-4 | cf. 8-3 with GML | 1.92 ± 0.58 |
| 8-5 | cf. 8-3 with GMS | 1.58 ± 0.24 |
| 8-6 | cf. 8-3 with GML + LL | 2.66 ± 0.67 |
| 8-7 | valdecoxib, silicone adhesive | 0.96 ± 0.14 |
| 8-8 | cf. 8-7 with GML | 1.18 ± 0.17 |
| 8-9 | cf. 8-7 with GMS | 0.84 ± 0.30 |
| 8-10 | cf. 8-7 with GML + LL | 1.38 ± 0.47 |
| 8-11 | parecoxib Na, PIB adhesive | 3.29 ± 3.05 |
| 8-12 | cf. 8-11 with GML | 11.28 ± 12.46 |
| 8-13 | parecoxib Na, acrylate adhesive | 3.36 ± 0.70 |
| 8-14 | cf. 8-13 with GML | 4.27 ± 2.06 |
| 8-15 | cf. 8-13 with GMS | 3.14 ± 1.10 |
| 8-16 | cf. 8-13 with GML + LL | ≦6.02 (n = 2) |
| 8-17 | parecoxib Na, silicone adhesive | 4.27 ± 1.68 |
| 8-18 | cf. 8-17 with GML | 4.35 ± 1.17 |
| 8-19 | cf. 8-17 with GMS | 3.60 ± 1.70 |
| 8-20 | cf. 8-17 with GML + LL | 3.89 ± 0.77 |
| 8-21 | parecoxib, PIB adhesive | 2.88 ± 0.43 |
| 8-22 | cf. 8-21 with GML | 3.48 ± 0.38 |
| 8-23 | parecoxib, acrylic adhesive | 7.03 ± 1.78 |
| 8-24 | cf. 8-23 with GML | 6.86 ± 0.58 |
| 8-25 | parecoxib, silicone adhesive | 10.44 ± 2.62 |
| 8-26 | cf. 8-25 with GML | 2.33 ± 0.82 |

The skin flux data in Table 9 are generally consistent with the findings reported in Table 6 above, although absolute levels of skin flux tended to be lower than in Table 6.

### Example 10

It was noted that in Example 9, certain patches did not adhere well to the skin membrane used in the permeation study. Therefore Compositions 8-3 to 8-6 and 8-13 to 8-20 were re-tested for skin permeation. Each patch was more firmly pressed on to the skin membrane than in the previous test, to ensure good adhesion. The skin membrane used in this study had higher permeability than that used for the same compositions in Example 9. Skin flux data are shown in Table 10.

TABLE 10
Skin flux of active agent from patches of Example 8

| Composition | Description | Skin flux (μg/cm² · day) |
|---|---|---|
| 8-3 | valdecoxib, acrylate adhesive | 4.8 ± 2.6 |
| 8-4 | cf. 8-3 with GML | 4.6 ± 1.0 |
| 8-5 | cf. 8-3 with GMS | 4.6 ± 0.7 |
| 8-6 | cf. 8-3 with GML + LL | 2.9 ± 0.5 |
| 8-13 | parecoxib Na, acrylate adhesive | 16.6 ± 2.2 |
| 8-14 | cf. 8-13 with GML | 19.9 ± 9.6 |
| 8-15 | cf. 8-13 with GMS | 17.5 ± 8.4 |
| 8-16 | cf. 8-13 with GML + LL | 30.7 ± 9.4 |
| 8-17 | parecoxib Na, silicone adhesive | 6.0 ± 1.2 |
| 8-18 | cf. 8-17 with GML | 15.4 ± 4.1 |
| 8-19 | cf. 8-17 with GMS | 19.2 ± 2.6 |
| 8-20 | cf. 8-17 with GML + LL | 25.2 ± 5.3 |

What is claimed is:

1. A pharmaceutical composition for application to an area of skin of a subject for local and/or systemic treatment of a COX-2 mediated disorder, the composition comprising a backing sheet that is flexibly conformable to the area of skin, said backing sheet having opposing surfaces that are respectively distal and proximal to the skin when applied; and a coating on the proximal surface of the backing sheet, said coating comprising (a) an adhesive and (b) a water-soluble active agent selected from the group consisting of selective COX-2 inhibitory drugs, prodrugs and salts thereof, the active agent being in a therapeutically effective total amount and dispersed in a matrix that comprises zero to less than an active agent solubilizing effective amount in total of one or more solvents other than the adhesive.

2. The composition of claim 1 wherein the active agent comprises a compound having the formula 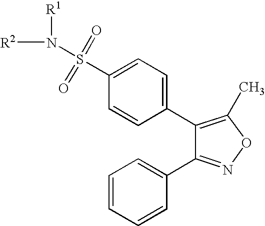
where R¹ and R² are independently hydrogen or a group that is metabolically replaceable by hydrogen; or a pharmaceutically acceptable salt of such a compound.

3. The composition of claim 2 wherein, in the formula for said compound, R¹ is hydrogen or a lower alkyl, hydroxyalkyl or acyl group and R² is hydrogen or a lower alkyl, hydroxyalkyl or acyl group or a group R³—CO— where R³ is hydrogen or a lower alkyl, lower alkoxy, lower carboxyalkyl, lower alkoxyalkyl, lower alkoxycarbonylalkyl, lower aminoalkyl, lower alkylcarbonylaminoalkyl, lower alkoxycarbonylaminoalkyl, phenyl or lower alkoxycarbonyl group.

4. The composition of claim 1 wherein the active agent comprises a compound having the formula 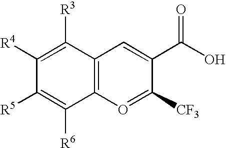
where R³ is hydrogen, halogen, or a lower alkyl, lower hydroxyalkyl or alkoxy group; R⁴ is hydrogen, halogen or a lower alkyl, lower alkylthio, lower haloalkyl, amino, aminosulfonyl, lower alkylsulfonyl, lower alkylsulfinyl, lower alkoxyalkyl, lower alkylcarbonyl, formyl, cyano, lower haloalkylthio, substituted or unsubstituted phenylcarbonyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5- or 6-membered heteroaryl, lower hydroxyalkyl, substituted or unsubstituted phenyl or 5- or 6-membered nitrogen-containing heterocyclosulfonyl group; R⁵ is hydrogen or halogen or a lower alkyl, lower haloalkyl, lower alkoxy or phenyl group; and R⁶ is hydrogen, halogen or a cyano, hydroxyiminomethyl, lower hydroxyalkyl, lower alkynyl, phenylalkynyl, lower alkyl, lower alkoxy, formyl or phenyl group; or a salt thereof.

5. The composition of claim 1 wherein the active agent comprises (S)-6,8-dichloro-2-(trifluoromethyl)-2H-1-benzopyran-3-carboxylic acid or a pharmaceutically acceptable salt thereof.

6. The composition of claim 1 wherein the active agent comprises (S)-6-chloro-7-(1,1-dimethylethyl)-2-(trifluoromethyl)-2H-1-benzopyran-3-carboxylic acid or a pharmaceutically acceptable salt thereof.

7. The composition of claim 1 wherein the coating comprises a layer having the active agent dispersed in a matrix that comprises the adhesive.

8. The composition of claim 7 wherein the active agent is dispersed at least partly in solid particulate form in the matrix.

9. The composition of claim 7 wherein the active agent is at least partly molecularly dispersed in the matrix.

10. The composition of claim 1 wherein the coating comprises a reservoir layer that comprises the active agent adjacent to the backing sheet, and an adhesive layer that is proximal to the skin when applied.

11. The composition of claim 10, further comprising a membrane between the reservoir layer and the adhesive layer, said membrane permitting passage of the active agent.

12. The composition of claim 1 wherein the coating further comprises at least one skin permeation enhancer.

13. The composition of claim 12 wherein the at least one skin permeation enhancer is selected from the group consisting of terpenes, terpenoids, fatty alcohols and derivatives thereof.

14. The composition of claim 12 wherein the at least one skin permeation enhancer is selected from the group consisting of oleyl alcohol, thymol, menthol, carvone, carveol, citral, dihydrocarveol, dihydrocarvone, neomenthol, isopulegol, 4-terpinenol, menthone, pulegol, camphor, geraniol, α-terpineol, linalool, carvacrol, trans-anethole, isomers thereof and racemic mixtures thereof.

15. The composition of claim 12 wherein the at least one skin permeation enhancer is selected from the group consisting of fatty acids and alkyl and glyceryl esters thereof.

16. The composition of claim 12 wherein the at least one skin permeation enhancer is selected from the group consisting of isopropyl laurate, isopropyl myristate, methyl oleate, glyceryl monolaurate, glyceryl monooleate, glyceryl monostearate, glyceryl dilaurate and glyceryl dioleate.

17. The composition of claim 12 wherein the at least one skin permeation enhancer is glyceryl monolaurate.

18. The composition of claim 12 wherein the at least one skin permeation enhancer is selected from fatty acid esters of glycolic acid and its salts.

19. The composition of claim 12 wherein the at least one skin permeation enhancer is selected from lactate esters of fatty alcohols.

20. The composition of claim 12 wherein the at least one skin permeation enhancer is selected from the group consisting of laurocapram and derivatives thereof, dimethylsulfoxide, n-decyl methylsulfoxide, salicylic acid and alkyl esters thereof, N,N-dimethylacetamide, dimethylformamide, N,N-dimethyltoluamide, 2-pyrrolidinone and N-alkyl derivatives thereof, 2-nonyl-1,3-dioxolane, eucalyptol and sorbitan esters.

21. The composition of claim 12 wherein the coating comprises about 1% to about 10% by weight of a water-soluble active agent selected from the group consisting of selective COX-2 inhibitory drugs, prodrugs and salts thereof, about 2% to about 20% by weight in total of one or more skin permeation enhancers, and about 70% to about 97% by weight of an adhesive composition.

22. The composition of claim 1 that further comprises a peelable release liner that, prior to use, is adjacent to the layer that contains the adhesive.

23. A method of local treatment of pain and/or inflammation at a site thereof in a subject, the method comprising a step of applying a pharmaceutical composition to a skin surface of the subject, said composition comprising a backing sheet that is flexibly conformable to the area of skin, said backing sheet having opposing surfaces that are respectively distal and proximal to the skin when applied, and a coating on the proximal surface of the backing sheet, said coating comprising (a) an adhesive and (b) a water-soluble active agent selected from the group consisting of selective COX-2 inhibitory drugs, prodrugs and salts thereof, the active agent being in a therapeutically effective total amount and dispersed in a matrix that comprises zero to less than an active agent solubilizing effective amount in total of one or more solvents other than the adhesive; and a step of leaving the composition in place for a time period effective to permit delivery of a locally therapeutic amount of the active agent.

24. The method of claim 23 wherein the skin surface to which the composition is applied is at a locus overlying or adjacent to the site of pain and/or inflammation.

25. A method of systemic treatment of a subject having a COX-2 mediated disorder, the method comprising a step of applying a pharmaceutical composition to a skin surface of the subject, said composition comprising a backing sheet that is flexibly conformable to the area of skin, said backing sheet having opposing surfaces that are respectively distal and proximal to the skin when applied, and a coating on the proximal surface of the backing sheet, said coating comprising (a) an adhesive and (b) a water-soluble active agent selected from the group consisting of selective COX-2 inhibitory drugs, prodrugs and salts thereof, the active agent being in a therapeutically effective total amount and dispersed in a matrix that comprises zero to less than an active agent solubilizing effective amount in total of one or more solvents other than the adhesive; and a step of leaving the composition in place for a time period effective to permit transdermal delivery of a therapeutic amount of the active agent.
